(19) United States
(12) Patent Application Publication
(10) Pub. No.: US 2024/0416151 A1
(43) Pub. Date: Dec. 19, 2024

(54) INTEGRALLY-STRUCTURED COMPLEX ULTRASOUND GENERATION TRANSDUCER

(71) Applicant: METASONIC INC., Yongin-si (KR)

(72) Inventors: Junghyun NAM, Seoul (KR); Moon Seop LEE, Siheung-si (KR); Myeong Woo LEE, Cheongju-si (KR); Kyung Han MIN, Suwon-si (KR)

(21) Appl. No.: 18/690,676

(22) PCT Filed: Sep. 13, 2022
(86) PCT No.: PCT/KR2022/013595

§ 371 Date: Mar. 8, 2024

(30) Foreign Application Priority Data
Sep. 14, 2021 (KR) 10-2021-0122744

(51) Int. Cl. A61N 7/02; A61N 7/00

(52) U.S. Cl. CPC A61N 7/02; A61N 2007/0034; A61N 2007/0073; A61N 2007/0078; A61N 2007/0091

(57) ABSTRACT

The present invention provides a complex ultrasound generation transducer that integrates the outputs of a multi-frequency generation transducer and a high-intensity focused ultrasound (HIFU) generation transducer, wherein the ultrasound generation transducer is linear, has a planar ultrasound generation section and a hemispherical ultrasound generation section formed on the upper surface of a cap-shaped body having a flat head, has a receiving portion formed as a concave circular groove on the lower surface of the upper portion, and has a piezoelectric element mounted inside the receiving portion.

### TECHNICAL FIELD

The present invention relates to an integrally-structured complex ultrasound generation transducer.

### BACKGROUND ART

An ultrasound transducer is a device that converts electrical energy into ultrasounds, which are thermal energy and mechanical energy, and are widely used in the industrial, medical and beauty fields. In particular, ultrasound is harmless and safe to the human body and therefore, much research is being conducted on the use of ultrasound technology not only in the medical field but also in the beauty field.

Recently, with the development of aesthetic medicine using ultrasound, interest in wrinkle reduction, skin strengthening, and aging prevention is increasing, and also demand for skin care products that utilize ultrasound to treat atopic dermatitis, acne scars, keloids, and eczema in the field of dermatology is increasing.

Multiple ultrasound transducers used as medical and beauty devices affect even the smallest particles inside cells with ultrasound frequencies that have an amplitude smaller than the skin cell wall thickness based on the principle of cross-generating multiple frequencies, reach up to a deep portion of the skin, and maximize the effects of skin regeneration through ultrasonic stimulation. High-intensity focused ultrasound transducers converge ultrasound and utilize intense ultrasound energy non-invasively to medically treat lesions within the body without the need for general anesthesia, aesthetically induces new collagen synthesis in the skin, and are used for skin tightening and lifting effects. Therefore, currently commercialized multi-frequency ultrasound transducers, also known as water droplet lifting or LDM, and high-intensity focused ultrasound (HIFU) transducers are used differently as transducers in a different manufacturing manner.

As a patent literature, Korean Patent No. 10-1672631 discloses a high-intensity focused ultrasound apparatus having a structure in which a cartridge unit includes an ultrasound focused element module and a linear piezoelectric motor, and a vibration unit including a vibration motor is coupled to the vibration cartridge unit, and a main body part electrically connected to the vibration unit includes a circuit unit and an outer power source unit.

Korean Patent No. 10-2117655 disclosure an LDM transducer comprising: a case formed with a storage space; a piezoelectric ceramic array consisting of a first piezoelectric body generating ultrasonic vibration of a first amplitude by electrical signals of first and second frequencies, and a second piezoelectric body laminated on the first piezoelectric body to generate ultrasonic vibrations of a second amplitude different from the first amplitude. The thickness of the first piezoelectric body is relatively thicker than the thickness of the second piezoelectric body.

In addition, Korean Patent No. 10-2221798 discloses an LDM-type transducer that simultaneously creates multiple aesthetic treatment zones in tissue. Korean Patent No. 10-2256560 discloses a HIFU type ultrasound generation device capable of adjusting the ultrasound focusing depth.

However, an integrally-structured complex ultrasound generation transducer that simultaneously generates multiple ultrasounds and high-intensity focused ultrasound waves has not been commercialized, and none of the patent literatures disclose it.

The present inventors have discovered that by changing the design of the ultrasound generation section, a complex ultrasound transducer having the whole functions of two ultrasound generators can be achieved.

### DETAILED DESCRIPTION OF THE INVENTION

### Technical Problem

It is an object of the present invention to provide a new transducer in which the effects of two or more transducers mentioned in the section above are simultaneously generated in one transducer, and the durability and stability are improved compared to the existing one.

### Technical Solution

In order to achieve the above object, according to one aspect of the present invention, there is provided a complex ultrasound generation transducer that integrates the outputs of a multi-frequency generation transducer and a high-intensity focused ultrasound (HIFU) generation transducer, wherein the ultrasound generation transducer includes a piezoelectric element whose material and vibration coefficient are determined using a transverse (thickness direction) oscillating ultrasonic drive frequency, and the maximum ultrasound is generated in the (4n-1) 24 region of the vibration period generated at the output end of the piezoelectric element, and wherein the ultrasound generation section of the ultrasonic generation transducer includes a first region consisting of a plurality of hemispherical shapes having a predetermined curvature, and a second region consisting of a planar shape.

The first region may be a region that generates high-intensity focused ultrasound, and the second region may be a region that generates multi-frequency ultrasound.

The material of the ultrasound generation section may include a metal material or an epoxy material.

The shape of the ultrasound generation section may be circular, rod-like, or polygonal, and the first region may consist of a plurality of circular, polygonal, arcuate, or conical shapes.

The focusing distance of the ultrasound may be changed by changing the diameter and curvature of each hemisphere in the first region of the ultrasound generation section.

The hemisphere of the first region may be arranged in a cross shape, and the peripheral portion may be arranged to form an arc.

The piezoelectric element may have a structure in which a plurality of piezoelectric elements are stacked and arranged.

The ultrasound generation section can move forward or backward.

According to another aspect of the present invention, there is provided a complex ultrasound generation transducer that integrates the outputs of a multi-frequency generation transducer and a high-intensity focused ultrasound (HIFU) generation transducer, wherein the ultrasound generation transducer is linear, has a planar ultrasound generation section and a hemispherical ultrasound generation section formed on the upper surface of a cap-shaped body having a flat head, has a receiving portion formed as a concave circular groove on the lower surface of the upper portion, and has a piezoelectric element mounted inside the receiving portion.

The maximum ultrasound may be generated in the (4n-1) 24 region of the vibration period generated at the output end of the piezoelectric element.

According to yet another aspect of the present invention, there is provided a complex ultrasound generation transducer that integrates the outputs of a multi-frequency generation transducer and a high-intensity focused ultrasound (HIFU) generation transducer, wherein: the ultrasound generation transducer includes a piezoelectric element whose material and vibration coefficient are determined using a transverse (thickness direction) oscillating ultrasonic drive frequency, the maximum ultrasound is generated in the (4n-1) 2/4 region of the vibration period generated at the output end of the piezoelectric element, the ultrasound generation section of the ultrasonic generation transducer includes a first region consisting of a plurality of hemispherical shapes having a predetermined curvature, and a second region in which the remaining portion except the first region has a planar shape, the first region is a region that generates high-intensity focused ultrasound, the second region is a region that generates multi-frequency ultrasound, high-intensity focused ultrasound generates multiple peak frequencies by each hemisphere of the first region, each peak frequency is the resonance frequency, and the impedance has a maximum and minimum value near each resonance frequency, resulting in high transmission and reception wave sensitivity.

### Advantageous Effects

The present invention solves the problem of having to implement different devices by different transducers depending on the existing ultrasonic generation type, outputs ultrasound energy from a single transducer simultaneously, sequentially, or in combination with multiple ultrasound and focused ultrasound, can uniformly transmit existing two-dimensional ultrasound energy to various types of skin in a three-dimensional vector form to the inner layer of skin, and can effectively act on skin and adipose connective tissue.

### BRIEF DESCRIPTION OF THE DRAWINGS

FIG. 1 is a diagram showing an ultrasound transducer according to an embodiment of the present invention that simultaneously generates multiple ultrasound and focused ultrasound.

FIG. 2 is a photograph showing an ultrasound transducer actually manufactured based on FIG. 1.

FIG. 3 is an example for explaining the actual object and the ultrasound characteristic principles of the multiple ultrasound transducer of the conventional technique.

FIG. 4 is an example for explaining the actual object and the ultrasound characteristic principles of a high-intensity focused ultrasound transducer of the conventional technique.

FIG. 5 shows ultrasound characteristic measurement data of a multiple ultrasound transducer implemented using the conventional technique of FIG. 3.

FIG. 6 shows ultrasound characteristic measurement data of the focused ultrasound transducer implemented using the conventional technique of FIG. 4.

FIG. 7 shows ultrasound output characteristic measurement data of the complex ultrasound transducer manufactured using the present invention shown in FIG. 2.

FIG. 8 is a graph showing ultrasound vibration characteristics according to frequency of the transducer manufactured according to the present invention of FIG. 2.

### DETAILED DESCRIPTION OF THE EMBODIMENTS

The objects and effects of the present invention and technical structures to achieve them will become apparent to those having ordinary skill in the art upon examination of the following embodiments of the present invention described with reference to the attached drawings. However, in the following description, a detailed description of known functions or constructions will be omitted lest it should obscure the subject matter of the present invention.

Throughout the description, when a portion is referred to as “including” or “comprising” a certain component, it means that the portion can further include other components, without excluding the other components, unless otherwise stated. Meanwhile, in an embodiment of the present invention, each component, functional block, or means may be composed of one or more sub-components.

Recently, various medical and cosmetic procedures for the purpose of skin regeneration treatment and beauty that utilize the characteristics of ultrasound have been researched and developed, and various types of ultrasonic beauty devices have emerged.

The main ultrasound technology implementation methods developed to date are two methods: a method that transmits single and multiple ultrasounds, and a focused ultrasound method (HIFU) that non-invasively focuses the ultrasound output, wherein ultrasound is transmitted to the human body using transducers with different structures.

Single and multiple ultrasound transducers stabilize the skin structure without damage by adjusting the fine tremors and penetration depth of intracellular and extracellular substances according to the drive frequency, but they does not generate a large amount of ultrasound energy, and therefore, the treatment must be performed for a long period of time, and the application location of ultrasound is limited. In addition, conventional focused ultrasound transducers usually have a structure that focuses and generates one ultrasound with a fixed curvature, and thus, when applied to a large skin area, they have the disadvantage of requiring a lot of treatment time due to mechanical movement of the focusing position. Further, there is a disadvantage that multiple cartridges must be used depending on the depth of focused ultrasound treatment.

In order to innovate the problems and limitations of existing ultrasound apparatuses, the present invention has newly analyzed and implemented the mechanical design of the output amplification unit so that it has the feature of generating multiple ultrasound and focused ultrasound at the same time, thereby implementing a new single-structure complex ultrasound transducer that can have diverse ultrasound output characteristics.

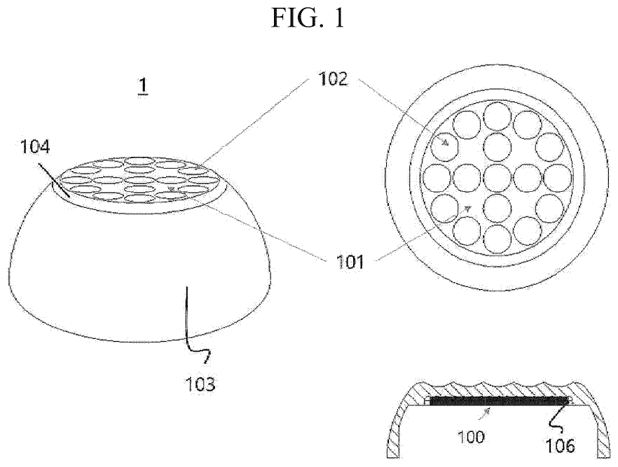

FIG. 1 is a diagram of the ultrasound transducer 1 of the present invention that simultaneously generates multiple ultrasound and focused ultrasound, wherein 100 indicates a piezoelectric element that generates an ultrasonic wave, 101 indicates a planar ultrasonic generation section, and 102 indicates a hemispherical ultrasonic generation section with a curvature.

The ultrasound transducer 1 has a streamlined outer shape, and consists of a cap-shaped main body 103 with a flat head. A planar ultrasound generation section 101 and a hemispherical ultrasound generation section 102 are formed on the upper surface of the upper part 104 of the main body 103, a receiving part 106 consisting of a concave circular groove is placed on the lower surface of the upper part 104, and a piezoelectric element 100 is mounted inside the receiving portion 106.

The hemispherical ultrasound generation section is made as a concave hemispherical groove with a predetermined curvature, but can also be made in an oval, square, or diamond shape. Further, the radius of curvature can also be set differently depending on the frequency of ultrasound. In the illustrated example, hemispherical arcs are arranged in a cross shape, and are arranged at close intervals surrounding the inner peripheral edge of the upper portion 104, which is just one example, and various shapes and pattern arrangements are possible.

Depending on the drive frequency according to the characteristics of the piezoelectric element that generates an ultrasonic wave, and the material and thickness of the ultrasound generation section that transmits and amplifies an ultrasonic wave, the maximum ultrasound driving generates on the upper surface of the ultrasound generation sections 101 and 102, which include hemispherical and planar shapes with single or multiple curvatures. In the present invention, high-intensity focused ultrasound is generated depending on the thickness of the upper surface of the ultrasonic generation section and the diameter and curvature of the hemisphere, and the planar ultrasound generation section 101 is a new structure that generates multiple ultrasonic waves traveling in a straight line. The material of the ultrasound generation sections 101 and 102 may include a metal material or an epoxy material.

The shape of the upper portion 104 on which the ultrasound generation sections 101 and 102 are mounted may be circular, rod-shaped, or polygonal.

Further, the hemispherical ultrasound generation section 102 may be formed not only in a hemisphere but also formed in circular, polygonal, arcuate, or conical shapes.

The present invention is characterized by the ultrasound transducer shown in FIG. 1, wherein as other members mounted to the body 103-a circuit containing a fluid for backing and frequency adjustment, an electrode connected to a piezoelectric element, and a power source, any of those known in the art can be adopted and utilized. In addition, a structure in which the body 103 is moved forward or backward can be adopted as in Korean Patent No. 10-2256560.

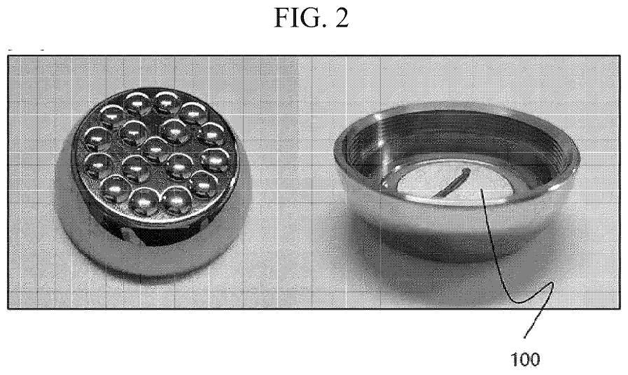

FIG. 2 is a diagram showing an actual product of the ultrasonic transducer 1 of FIG. 1.

The driving frequency to be used for the piezoelectric element 100 and the diameter and thickness of the hemisphere were calculated and a desired product was manufactured through simulation. As for the material of the ultrasound generation section, the thickness of the ultrasound generation section was determined by reflecting the vibration coefficients of metal and epoxy. This is an embodiment in which a piezoelectric element 100, which generates an ultrasonic wave by forming a hemispherical and planar shape with a curvature on the upper portion 104, and an ultrasound generation section, which amplifies and transmits an ultrasonic wave, are actually combined and attached.

According to the present invention, when ultrasonic waves with single and multiple drive frequencies are generated, the ultrasonic wave can be transmitted to the human body and skin at different depths depending on the drive frequencies. Two ultrasound transducers capable of adjusting the ultrasound output intensity by focusing ultrasonic waves in multiple directions and adjusting the position up to the skin surface and the deep portion inside the human body are realized as an integrally-structured ultrasound transducer 1. Therefore, by combining the advantages of the conventional multi-frequency ultrasound device and the advantages of the high-intensity focused ultrasound device and also complementing the limitations of each device, safe procedures for the human body and skin can be performed, and the durability and accuracy of the transducer are improved.

The piezoelectric element that generates ultrasonic waves has a drive frequency constant (Nr) for radial (circumferential) vibration and a drive frequency constant (Nt) for transverse (thickness direction) vibration, wherein these constants vary depending on the piezoelectric materials, and varies depending on the thickness of the piezoelectric element. The drive range if the integers includes multiple vibration frequencies rather than a single frequency, and ultrasonic waves can be generated at several frequencies using single and multiple vibration frequencies.

Since the transducers in the present invention apply ultrasonic waves to the deep portion into the human body and skin, the transverse vibration ultrasonic drive frequency of the piezoelectric element 100 is determined and used.

The ultrasound generation section, which plays the role of transmitting and amplifying the ultrasonic waves generated by the piezoelectric element 100, is related to the drive frequency at which ultrasonic waves are generated and the vibration coefficient and thickness depending on the material of the generation section to which the ultrasonic waves are transmitted. Vibration starts at the center of the piezoelectric element 100, and the material and thickness are determined and calculated using the vibration coefficient of the ultrasound generation section combined and attached to the vibration coefficient of the piezoelectric element 100. The (4n−1) λ/4 region of the vibration period generated at the output end of the piezoelectric element 100 is the point where the maximum ultrasound is generated. This geometric position is designed and configured by combining a planar shape of the upper surface of the ultrasound generation section and a hemispherical shape including curvature, thereby realizing a single-structure complex ultrasonic generation transducer having simultaneous output characteristics of complex ultrasound and focused ultrasound. Here, n is the vibration constant (1, 2, 3, 4 . . . ).

In the present invention, the piezoelectric element 100 is described as having a disk shape, but a donut shape or a cylinder shape with an empty center is also possible. Further, the piezoelectric element 100 can be manufactured by stacking multiple layers of piezoelectric elements 100.

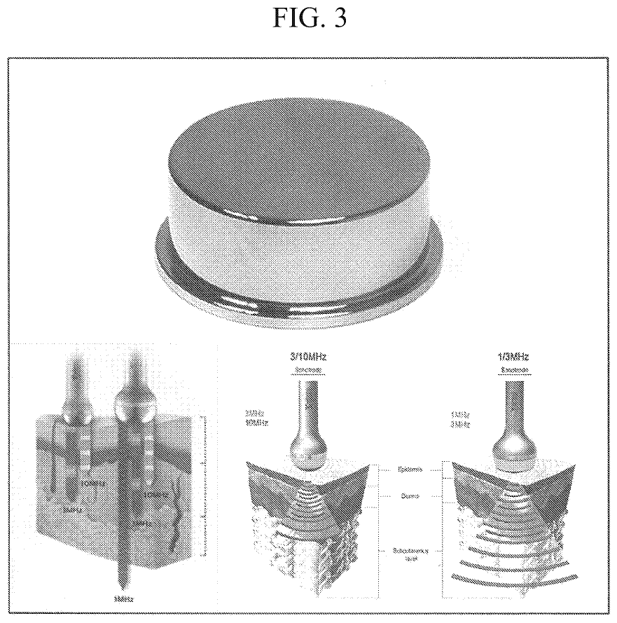

FIG. 3 is an actual photograph of the multiple ultrasound transducer 1a of the conventional technique and an example for explaining the characteristics of ultrasound penetrating into the human body and skin. Since the transducer 1a cross-generates an ultrasonic wave with different vibrations by crossing multiple drive frequencies as shown in FIG. 3, it causes stable changes in various skin cells, including the effect of stabilizing the structure of the human body and skin by adjusting the ultrasonic vibration of the human body and skin and the depth within the human body without damaging the skin.

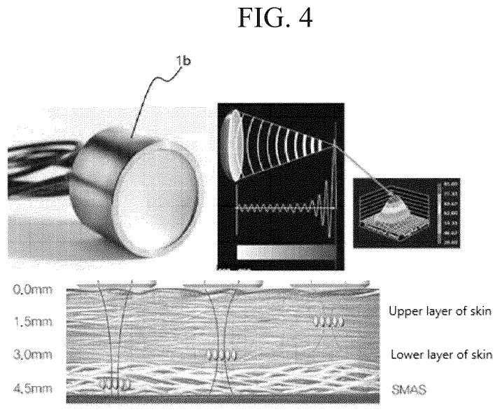

FIG. 4 is an actual photograph of the conventional high-intensity focused ultrasound transducer 1b and an example for explaining the principle of the focused ultrasound characteristics and the penetration position within the skin. The transducer 1b focuses ultrasonic waves generated in multiple directions within its area and amplifies the generated energy to generate the maximum ultrasonic output. Using focused ultrasound, it treats the human body and skin without external wounds and generates biological changes in skin regeneration and lifting.

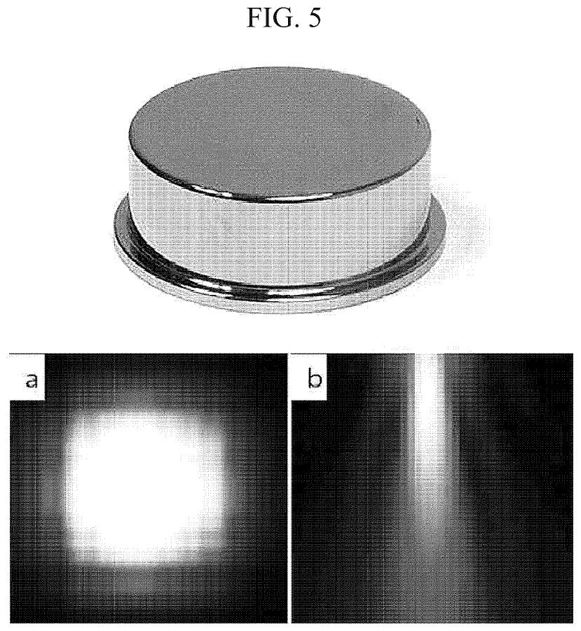

FIG. 5 shows data measured with a hydrophone as the ultrasound characteristic measurement distribution of the multiple ultrasound transducer 1a implemented using the conventional technique of FIG. 3. A hydrophone audible unit, that is, a hydrophone, is a device that measures the generation direction, output intensity, and distribution of ultrasonic waves, and confirms the ultrasound generation output data with the naked eye. In FIG. 5 (a), it can be seen that ultrasonic waves are generated with similar output intensity across the entire upper surface, which is the ultrasound generation section. As shown in FIG. 5 (b), the ultrasonic region is represented by a distribution in which the output is transmitted starting from the upper surface and the output intensity becomes weaker as the depth increases.

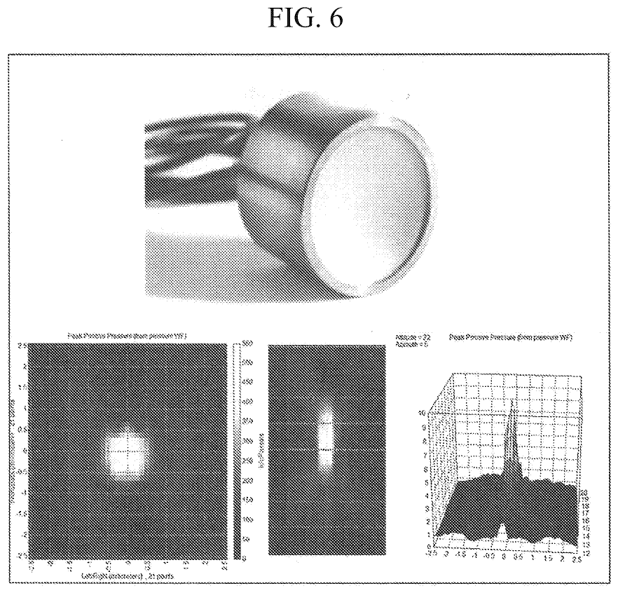

FIG. 6 is a measurement distribution of ultrasonic characteristics of the high-intensity focused ultrasonic transducer (1b) implemented using the conventional technique of FIG. 4, which is data measured with a hydrophone, as shown in FIG. 5. In FIG. 6a, the strongest distribution of ultrasonic output intensity was confirmed at the center of the transducer, and it can be confirmed that the ultrasonic output intensity is strong at a distance from the upper surface depending on the diameter and curvature of the transducer as shown in FIG. 6b. When the three-dimensional graph is confirmed in FIG. 6c, the curvature focuses multi-directional ultrasound waves at one location and exhibits the maximum ultrasound output area at the desired depth.

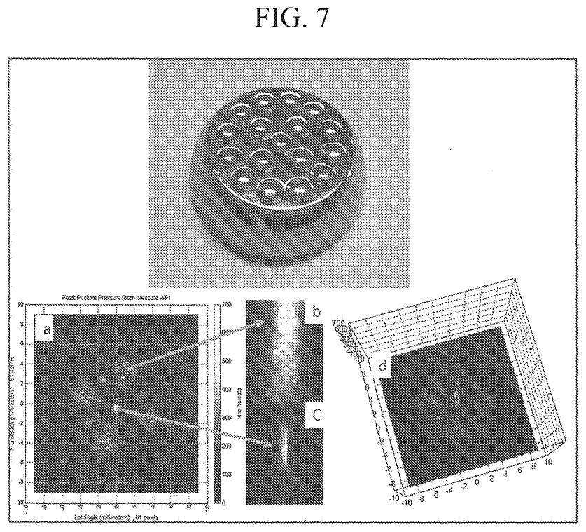

FIG. 7 shows data measured using a hydrophone for ultrasonic characteristic measurement and testing of the ultrasonic transducer 1 of the present invention under the same conditions as in the conventional ultrasonic characteristic measurement in FIGS. 5 and 6, which is an example using the actual product of the present invention. As shown in the photograph of FIG. 7, it can be confirmed that the upper surface, which is the ultrasound generation section, has a hemispherical shape with a curvature that generates focused ultrasonic waves and a plane shape that generates multiple ultrasonic waves.

FIG. 7a shows the ultrasound intensity distribution on the upper surface, which confirms that focused ultrasonic waves are generated in a hemispherical shape, and multiple ultrasonic waves are generated in a planar shape. FIG. 7b is multiple ultrasonic generation data in a planar shape. It can be seen that output starts from the upper surface as shown in FIG. 5b. FIG. 7c shows focused ultrasound generation data in a hemispherical shape with curvature, and it can be confirmed that ultrasonic waves are focused at a certain depth from the upper surface as shown in FIG. 6b. FIG. 7c simultaneously shows focused ultrasound characteristics as shown in FIG. 6 in a hemispherical shape having a three-dimensional curvature and multiple ultrasound characteristics across a planar shape as shown in FIG. 5.

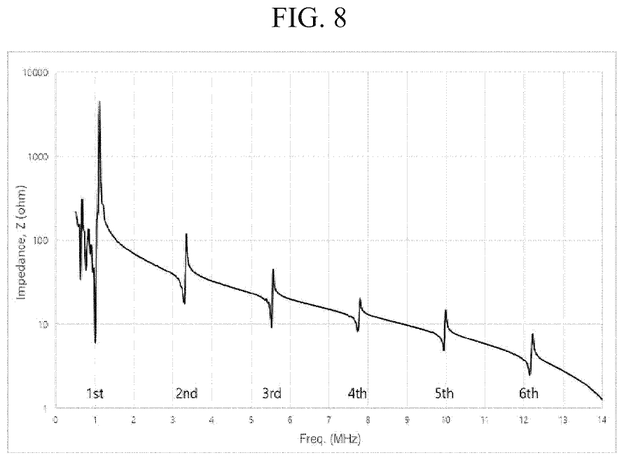

FIG. 8 is a diagram showing ultrasound vibration characteristics according to frequency of the transducer 1 according to the embodiment of the present invention in FIG. 7. As shown in FIG. 7, the drive points of two ultrasonic characteristics are explained. Each peak frequency is a resonance frequency, and a variety of choices are possible, for example, the thickness and physical properties of the piezoelectric element 100 and the curved surface shape and position of the ultrasonic wave generating section are adjusted to generate, for example, the first and third resonance frequencies (1 MHZ, 3.2 MHz). The transmitted wave sensitivity is maximum at the resonance frequency where impedance is minimum, and the received wave sensitivity is maximum at the anti-resonance frequency where impedance is maximum. In the case of the present invention, it can be confirmed that since the impedance has a maximum value and a minimum value near each resonance frequency, the transmission wave sensitivity is excellent.

The present invention described above is a new type of ultrasonic transducer that realizes the characteristics of various existing ultrasonic transducers into a single ultrasonic transducer, and is characterized by implementing the generation of multi-driven ultrasonic waves and focused ultrasonic waves in an integrated manner, simplifying the structure, and enabling simultaneous or sequential output of the characteristics of several ultrasonic waves.

The ultrasonic transducer structure of the present invention includes a piezoelectric element 100 that generates a plurality of ultrasonic waves, and an upper surface configured to generate amplified and transmitted multiple ultrasonic waves and focused ultrasonic waves. Due to such a structure, multiple ultrasonic waves are generated on the entire surface of the upper surface, and at the same time, the focal distance and focusing area of the focused ultrasonic waves can be varied depending on the design of the hemispherical curvature and diameter of the upper surface, making it possible to generate the desired focused ultrasonic waves.

Therefore, the present invention solves the problem of existing ultrasound generation types that need to be performed with different transducers and different equipment, and allows multiple ultrasound waves and focused ultrasound waves to be generated simultaneously or sequentially in a single transducer, whereby the existing two-dimensional ultrasound energy can be transferred homogeneously to the inner layer of skin in a three-dimensional vector type to various types of skin, thereby effectively acting on the skin and adipose connective tissue.

It would be obvious that the scope of rights of the present invention extends to the same or equivalent region as the scope of the claims described below.

1. A complex ultrasound generation transducer that integrates the outputs of a multi-frequency generation transducer and a high-intensity focused ultrasound (HIFU) generation transducer,
wherein the ultrasound generation transducer includes a piezoelectric element whose material and vibration coefficient are determined using a transverse (thickness direction) oscillating ultrasonic drive frequency, and the maximum ultrasound is generated in the (4n−1) λ/4 (where n is the vibration constant) region of the vibration period generated at the output end of the piezoelectric element, and
wherein the ultrasound generation section of the ultrasonic generation transducer includes a first region consisting of a plurality of hemispherical shapes having a predetermined curvature, and a second region consisting of a planar shape.

2. The complex ultrasound generation transducer of claim 1, wherein:
the first region is a region that generates high-intensity focused ultrasound, and the second region is a region that generates multi-frequency ultrasound.

3. The complex ultrasound generation transducer of claim 2, wherein:
the material of the ultrasound generation section includes a metal material or an epoxy material.

4. The complex ultrasound generation transducer of claim 2, wherein:
the shape of the ultrasound generation section is circular, rod-like, or polygonal, and the first region consists of a plurality of circular, polygonal, arcuate, or conical shapes.

5. The complex ultrasound generation transducer of claim 2, wherein:
the focusing distance of the ultrasound is changed by changing the diameter and curvature of each hemisphere in the first region of the ultrasound generation section.

6. The complex ultrasound generation transducer of claim 2, wherein:
the hemisphere of the first region is arranged in a cross shape, and the peripheral portion is arranged to form an arc.

7. The complex ultrasound generation transducer of claim 2, wherein:
the piezoelectric element has a structure in which a plurality of piezoelectric elements are stacked and arranged.

8. The complex ultrasound generation transducer of claim 2, wherein:
the ultrasound generation section can move forward or backward.

9. A complex ultrasound generation transducer that integrates the outputs of a multi-frequency generation transducer and a high-intensity focused ultrasound (HIFU) generation transducer,
wherein the ultrasound generation transducer is linear, has a planar ultrasound generation section and a hemispherical ultrasound generation section formed on the upper surface of a cap-shaped body having a flat head, has a receiving portion formed as a concave circular groove on the lower surface of the upper portion, and has a piezoelectric element mounted inside the receiving portion.

10. The complex ultrasound generation transducer of claim 9, wherein:
the maximum ultrasound is generated in the (4n−1) λ/4 region of the vibration period generated at the output end of the piezoelectric element.
